(19) United States
(12) Patent Application Publication
(10) Pub. No.: US 2005/0283230 A1
(43) Pub. Date: Dec. 22, 2005

(54) Heat switch

(76) Inventors: Chandrashekhar Joshi, Bedford, MA (US); Chiu-Ying Tai, Chelmsford, MA (US); Anil Mavanur, North Andover, MA (US)

Correspondence Address: CHOATE, HALL & STEWART LLP, TWO INTERNATIONAL PLACE, BOSTON, MA, 02110, US

(21) Appl. No.: 11/153,976

(22) Filed: Jun. 16, 2005

Related U.S. Application Data
(60) Provisional application No. 60/581,469, filed on Jun. 21, 2004.

(51) Int. Cl. A61F 2/06

(52) U.S. Cl. 623/1.44

(57) ABSTRACT

Heat switch. The switch includes a magnetostrictive member and a coil arranged to apply a magnetic field to the magnetostrictive member to cause the member to change from a first state to a second state of elongation. A heat conductive flexible structure is coupled to the magnetostrictive member so as to change its lateral extent in response to a change from the first state of elongation to the second state of elongation of the magnetostrictive member. A heat conductive housing is adjacent to at least one surface of the flexible structure so that the at least one surface of the flexible structure is in contact with the housing in one state of elongation of the magnetostrictive member and out of contact with the housing in the other state of elongation of the magnetostrictive member. The switch controls the flow of heat from a relatively warmer surface to a relatively colder surface.

### HEAT SWITCH

This application claims priority to provisional application Ser. No. 60/581,469 filed Jun. 21, 2004 and entitled “Compact Mechanical Heat Switch.” The contents of this provisional application are incorporated herein by reference in their entirety.

### BACKGROUND OF THE INVENTION

This invention relates to a switchable thermal link and more particularly to a compact mechanical heat switch.

Many systems need to be maintained at constant low temperatures for their successful operation. Devices that are used to maintain such temperatures must be cycled between the maintained temperature and the temperature of a heat sink to which heat is rejected. A switchable thermal link (“heat switch”) is required in such a cyclic cooling mechanism. By heat switch is meant a device that conducts heat in one state and which does not conduct heat in another state, in analogy with an electrical switch. An improvement in heat switch technology will directly lead to improvement of modern cooling systems and is therefore very important to extremely temperature sensitive sensors and instruments such as for space missions and magnetic resonance imaging. One of the most popular applications for heat switches is with adiabatic demagnetization refrigerators (ADRs).

Different types of the switchable thermal links have been developed including superconducting heat switches, gas-gap or liquid-gap heat switches, and mechanical heat switches. Mechanical heat switches are typically the most attractive of the heat switch devices based on their operating temperature range, the on/off ratio of the thermal conductance, and small power requirements.

FIG. 1. is a well known prior art “cold-finger” heat switch. See, C. Hagmann and P. L. Richards, “Adiabatic Demagnetization Refrigerators for the Small Laboratory Experiments and Space Astronomy”, Cryogenics, 35, pp. 303-309. (1995). In FIG. 1, energizing of a solenoid 10 lifts a ferromagnetic yoke 12 against a restoring spring 14 and through a linkage 16 closes jaws 18 on a cold finger 20 that may extend from a 100 mK stage. The closing of the jaws 18 on the cold finger 20 establishes a thermal link. Heat switches of this type include air piston driving devices and alternative cold finger designs having more than one finger.

It is known that the thermal conductance between two contact surfaces is strongly dependent on the contact force. The contact force between the jaws 18 and the cold finger 20 in the design shown in FIG. 1 relies on the geometry of the linkages 16 and the power of the solenoid 10. Achieving a high contact force will likely require an increase in the size and driving power of the mechanical heat switch. Apart from the size and power concerns with the heat switch design of FIG. 1, there are too many moving parts and joints that can cause maintenance problems in space missions. An object of the present invention is improved mechanical heat switch designs.

### SUMMARY OF THE INVENTION

In one aspect, the heat switch according to the invention includes a magnetostrictive member along with a coil arranged to apply a magnetic field to the magnetostrictive member so as to cause the member to change from a first state to a second state of elongation. A heat conductive flexible structure is coupled to the magnetostrictive member in a manner to change its lateral extent in response to a change from the first state of elongation to the second state of elongation of the magnetostrictive member. A heat conductive housing is adjacent to at least one surface of the flexible structure so that at least one surface of the flexible structure is in contact with the housing in one state of elongation of the magnetostrictive member and out of contact with the housing in the other state of elongation of the magnetostrictive member.

In one embodiment of the invention the flexible structure is in contact with the housing when the coil is de-energized. In another embodiment, the flexible structure is in contact with the housing when the coil is energized. The coil may be superconducting and it is preferred that the coil surround that the magnetostrictive member.

In a preferred embodiment, the flexible structure is a barrel-shaped, slotted body having ends affixed to ends of the magnetostrictive member. Alternatively, the flexible structure may be substantially rectangular with protruding contact surfaces. In yet another variation, the flexible structure is a concave surface of revolution with a protruding contact portion.

In still another embodiment, the flexible structure is in thermal contact with a relatively colder surface and the housing is in thermal contact with a relatively warmer surface so that heat will flow from the relatively warmer surface to the relatively colder surface when a surface of the flexible structure is in contact with the housing. Heat will not flow when a surface of the flexible structure is not in contact with the housing. It is preferred that the housing be in thermal contact with the relatively warmer surface through thermal straps. A suitable material for the flexible structure and the housing is dispersion strengthened copper or the copper with some nickel or magnesium. Suitable magnetostrictive materials include, but are not limited to, the general class of materials with compositions containing terbium, dysprosium and iron or zinc which include Terfenol, TbDyFe, TbDyZn or DyZn. The assignee of the present invention uses the trade name KevinAll for its composition of TbDyFe.

### BRIEF DESCRIPTION OF THE DRAWING

FIG. 1 is a cross-sectional view of a prior art mechanical heat switch.

FIG. 2 is a schematic illustration of a magnetostrictive actuator.

FIGS. 3a, b and c are an exploded view, top view and cross-sectional view, respectively, of an embodiment of the heat switch of the invention.

FIGS. 4a, b and c are a side view, top view and perspective view, respectively, of an embodiment of the invention.

FIGS. 5a and b are side views of an embodiment of the invention showing power off and power on, respectively.

FIGS. 6a and b are a perspective view and a side view respectively of another embodiment of the invention.

FIGS. 7a and 7b are a perspective view and a cross-sectional view, respectively, of yet another embodiment of the invention.

FIG. 8. is a cross-sectional view of another embodiment of the invention.

### DESCRIPTION OF THE PREFERRED EMBODIMENT

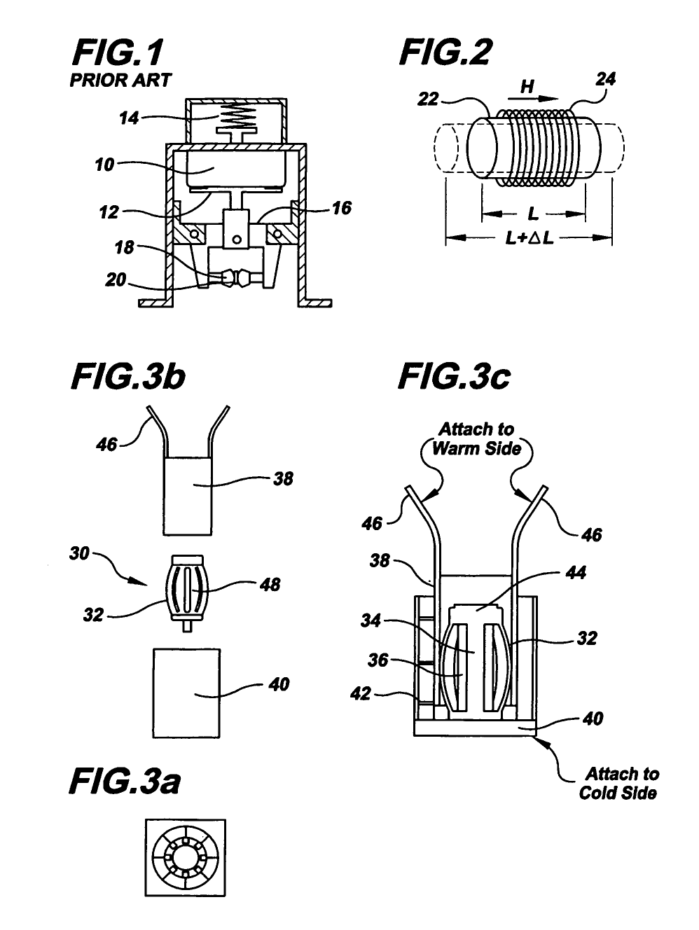

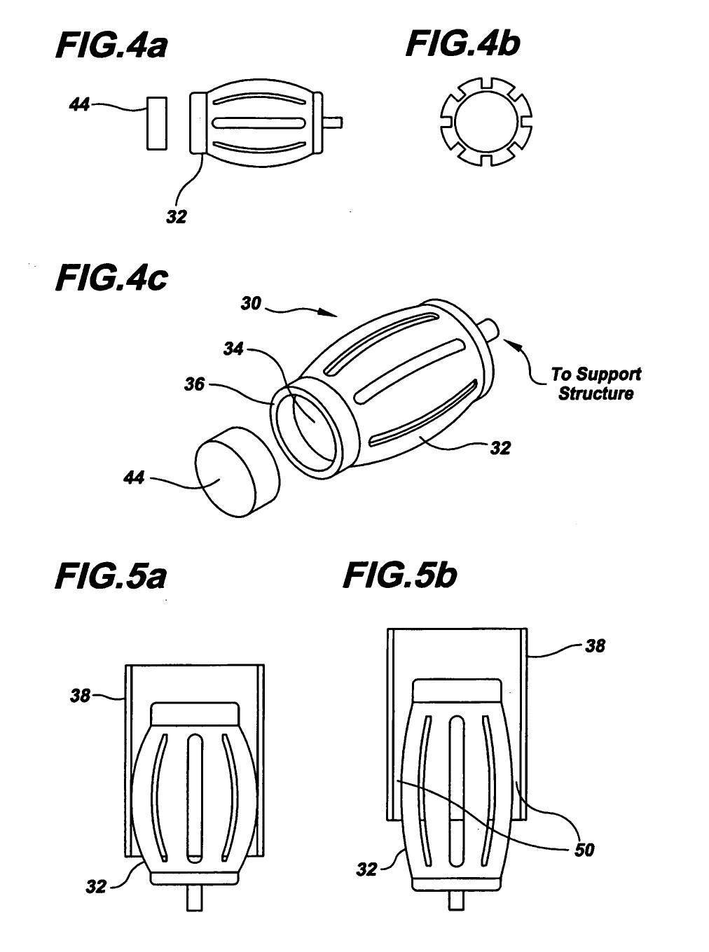

The present invention utilizes magnetic “smart” materials (MSM) or magnetostrictors, materials that change their shape when exposed to a magnetic field. Magnetostriction arises from a re-orientation of the atomic magnetic moments within magnetostrictive materials. These materials exhibit a reversible dimensional change in response to an externally applied magnetic field. As shown FIG. 2, a magnetostrictive rod 22 will elongate to a length L+Δ L in response to magnetic field H provided by a magnetic coil 24. Those skilled in the art will appreciate that the change in length of the rod 22 can be used as an actuator.

With reference now to FIGS. 3a-c and 4a-c, an actuator assembly 30 includes a barrel actuator 32 mechanically attached to a magnetostrictive rod 34. A coil 36 surrounds the rod 34 to provide an actuating magnetic field. The actuator assembly 30 is positioned within a cylindrical housing 38 and the cylindrical housing 38 is contained within a support structure 40 by means of Kevlar strings 42. An adjustable/removable end cap 44 is used to adjust the pre-loading force on the magnetostrictive rod 34, and to stretch the barrel 32 for assembly. Thermal straps 46 are connected to the cylindrical housing 38.

The cylindrical housing 38 has a smaller inner diameter than the maximum outer diameter of the barrel 32 in its rest state. For assembly, the barrel 32 is mechanically stretched to reduce its diameter so that it will fit within the cylindrical housing 38. When the barrel 32 and cylindrical housing 38 are in the appropriate position, the mechanical stretching is released so that a large contact force will result between the barrel 32 and the cylindrical housing 38. Slots 48 are provided in the barrel 32 to reduce its stiffness.

As those skilled in the art will appreciate, the thermal conductance (K) between two contact surfaces is strongly dependent on the contact force. Conductance of a few hundred mW/K has been reported with 1,000 N contact force between two gold-plated copper surfaces by L. J. Salerno and P. Kittel in “Thermal Contact Conductance,” NASA Ames Research Center, Calif. This paper may be accessed at htt://irtek.arc.nasa.gov/CryoGroup/Archive/LS_{—}98HB.word.

With reference now to FIG. 5a, the coil is not energized and the sides of the barrel actuator 32 are in intimate contact with the inner surface of the cylindrical housing 38. When the coil 36 is energized, the magnetostrictive rod 34 elongates thereby narrowing the barrel actuator 32 and creating a gap 50 between the barrel actuator 32 and the inner surface of the cylindrical housing 38.

As best seen in FIG. 3c, the support structure 40 is contacted to a cold side in a heat flow circuit and the thermal straps 46 are attached to a warm side. Therefore, when the switch of the invention is in the power off state as shown in FIG. 3c and FIG. 5a, heat will flow along the thermal straps 46 into the cylindrical housing 38, then into the barrel actuator 32 and through the support structure 40 to the cold side of a thermal flow path.

As shown in FIG. 5b, when the actuator is powered on the barrel 32 narrows and there is no contact between the barrel actuator 32 and the cylindrical housing 38 so that there is no longer a path for heat to flow through the heat switch.

As stated above, the cylindrical housing 38 is thermally connected to the warm side through several flexible thermal straps 46. This design provides the required thermal transition between the warm and cold sides while the heat switch of the invention is closed. In order to satisfy both thermal and mechanical requirements, suitable materials for the actuator and housing include dispersion strengthened copper and copper with very small amounts of nickel or magnesium.

The embodiment just described is appropriate for space applications. In order to avoid misalignment of the switch at launch caused by a relative position change of the cold and warm walls, the cylindrical housing 38 is physically connected to the support structure 40 using the Kevlar strings 42 as shown in FIG. 3c. The low thermal conductivity of the Kevlar strings that couple the cylindrical housing 38 to the support structure 40 ensures the required low thermal leak when the switch is open and the webbing arrangement shown schematically in FIGS. 3b and c provides strong enough support for the cylindrical housing 38 to prevent misalignment with the barrel actuator 32.

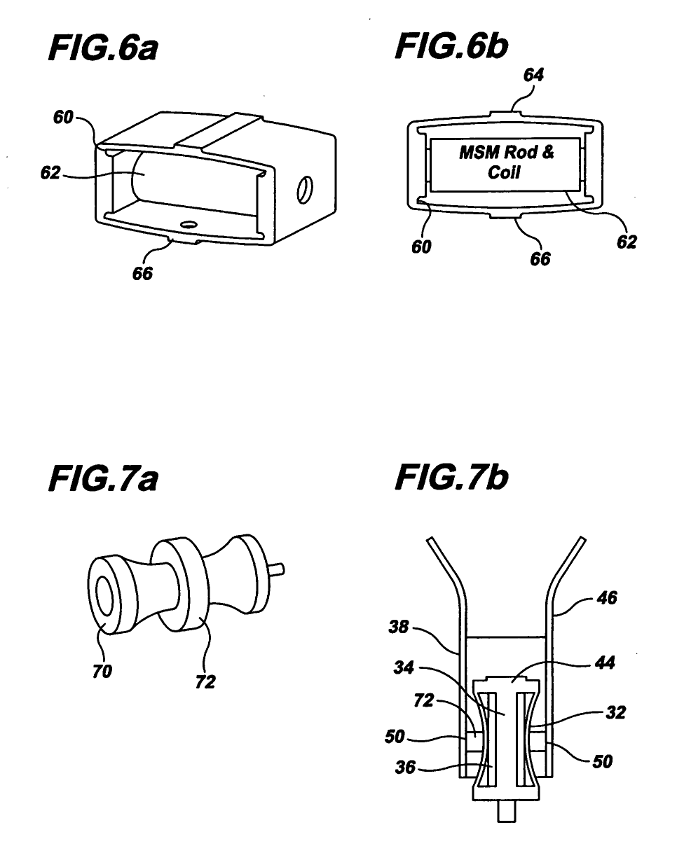

Another embodiment of the invention is shown in FIGS. 6a and 6b, an amplified actuator that provides larger output actuation than a magnetostrictive rod could create by itself. In the embodiment of FIG. 6, a rectangular flexible structure 60 encompasses a magnetostrictive rod and coil assembly 62. The structure 60 includes two contact surfaces 64 and 66. Those skilled in the art will appreciate that the structure 60 will be positioned within an appropriate housing to provide the requisite thermal pathway similar to the embodiment described above.

Yet another embodiment of the invention is shown in FIGS. 7a and b. In this embodiment an actuator 70 includes a surface of rotation with a protruding portion 72. Note that in this embodiment, a gap exists between the actuator and housing when the power is off and the actuator is in its rest state. When the coil 36 is energized the magnetostrictive rod 34 will elongate and force the protruding portion 72 into contact with the cylindrical housing 38 thereby closing the switch and providing a heat flow path.

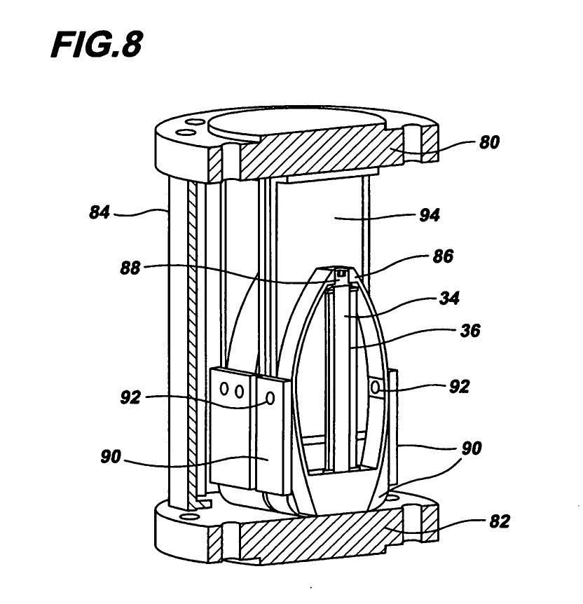

A still further embodiment is shown FIG. 8. The embodiment of FIG. 8 illustrates two identical actuators with parallel connection. For clarity, only half of a front actuator is seen in a cross-sectional view. A warm side flange 80 and cold side flange 82 are rigidly connected to one another by a low thermal conductivity support 84. The low thermal conductivity support 84 allows low thermal leak when the switch of this embodiment is “open”. The magnetostrictive member 34 is surrounded by the superconducting coil 36. The magnetostrictive member 34 and superconducting coil 36 reside in the center of an amplified actuator housing 86. An adjustable cap screw 88 is used to adjust the preload force on the magnetostrictive member 34 and to stretch the actuator housing 86.

The actuator housing 86 and a cold side contact piece 90 are thermally anchored to the cold side flange 82. The cold side contact piece 90 has its two side walls mechanically attached to the actuator housing 86 at the connecting points 92. The actuator housing 86 is preferably made of high yield strength material such as steel so that it is not fatigued under repeated flexing. The cold side contact piece 90 is made of high thermally conductive material such as copper, preferably with gold plated surfaces, so as to provide a high thermal conduction link to the cold side flange 82. A warm side contact piece 94 comprises two fins located in front of and behind the housing 86 and is thermally anchored to the warm side flange 80. The warm side contact piece 94 is also preferably made of copper with gold-plated surfaces. The entire unit just described is used in a chamber evacuated of air or any other gases to minimize any parasitic heat flow between the two flanges 80 and 82.

When power is off so that the superconducting coil 36 is not energized, there is a gap between the warm side contact fins 94 and the cold side contact piece 90. Thus, the switch is in the “open” state and substantially no heat flows from the warm side flange 80 to the cold side flange 82.

When power is turned on to energize the superconducting coil 36, the magnetic field causes the magnetostrictive member 34 to elongate. This elongation stretches the amplified housing 86 causing the cold side contact pieces 90 to come strongly into contact with the edges of the warm side contact fin 94. The high contact force between the cold side contact pieces 90 and the edges of the warm side contact fin 94 provides a high thermal conduction path between the warm side flange 80 and the cold side flange 82. The switch is thus “closed”.

The heat switch design of FIG. 8 with two parallel actuators will have twice the thermal conductance that each actuator alone could provide. While a two contact design has been illustrated, a single contact or more than two contacts can be used between the two flanges to achieve a desired thermal conductance.

Those skilled in the art will recognize that high permeability and high resistivity materials for flux concentration and magnetic shielding can be used. Configurations without flux concentration can also be utilized with different coil designs. It is also noted that the embodiments disclosed herein can be driven by ball screws and motors rather than by magnetostrictive rods and coils.

The heat switch designs disclosed above result in negligible physical deformation due to variations in outside temperature. These designs result in high force capability and rapid response. They operate with low voltages with a compact size and light weight.

It is recognized that modifications and variations of the present invention will occur to those of ordinary skill in the art and it is intended that all such modifications and variations be included within the scope of the appended claims.

1. Heat switch comprising:
a magnetostrictive member;
a coil arranged to apply a magnetic field to the magnetostrictive member to cause the member to change from a first state to second state of elongation;
a heat conductive flexible structure coupled to the magnetostrictive member so as to change its lateral extent in response to a change from the first state of elongation to the second state of elongation of the magnetostrictive member; and
a heat conductive housing adjacent to at least one surface of the flexible structure,
whereby the at least one surface of the flexible structure is in contact with the housing in one state of elongation of the magnetostrictive member and out of contact with the housing in the other state of the elongation of the magnetostrictive member.

2. The heat switch of claim 1 wherein the flexible structure is in contact with the housing when the coil is de-energized.

3. The heat switch of claim 1 wherein the flexible structure is in contact with the housing when the coil is energized.

4. The heat switch of claim 1 wherein the coil surrounds the magnetostrictive member.

5. The heat switch of claim 1 wherein the flexible structure is a barrel-shaped, slotted body having ends affixed to ends of the magnetostrictive member.

6. The heat switch of claim 1 wherein the flexible structure is substantially rectangular with protruding contact surfaces.

7. The heat switch of claim 1 wherein the flexible structure is a concave surface of revolution with a protruding contact portion.

8. The heat switch of claim 5 wherein a surface of the barrel-shaped body is in contact with the housing when the coil is de-energized and out of contact with the housing when the coil is energized.

9. The heat switch of claim 6 wherein the protruding contact surfaces are in contact with the housing when the coil is de-energized and out of contact with the housing when the coil is energized.

10. The heat switch of claim 7 wherein the protruding contact portion is in contact with the housing when the coil is energized and out of contact with the housing when the coil is de-energized.

11. The heat switch of claim 1 wherein the housing is cylindrical.

12. The heat switch of claim 1 wherein the flexible structure is in thermal contact with a relatively colder surface and the housing is in thermal contact with a relatively warmer surface, whereby heat will flow from the relatively warmer surface to the relatively colder surface when a surface of the flexible structure is in contact with the housing, and heat will not flow when a surface of the flexible structure is not in contact with the housing.

13. The heat switch of claim 5 further including a support structure surrounding the housing and supporting the housing through low thermal conductivity attachments.

14. The heat switch of claim 12 wherein the housing is in thermal contact with the relatively warmer surface through thermal straps.

15. The heat switch of claim 13 wherein the low thermal conductivity attachments comprise Kevlar strings.

16. The heat switch of claim 1 wherein the coil is a superconducting coil.

17. The heat switch of claim 1 wherein contact force between a surface of the flexible member and the housing is approximately 1,000 newtons.

18. The heat switch of claim 1 wherein the flexible structure and housing are made of dispersion strengthened copper or copper with some nickel or magnesium.

19. The heat switch of claim 1 wherein the magnetostrictive material contains terbium, dysprosium and iron or zinc.

20. The heat switch of claim 19 wherein the magnetostrictive material is selected from the group consisting of Terfenol-D, TbDyFe, TbDyZn or DyZn.

21. Heat switch comprising:
a magnetostrictive member;
a coil arranged to apply a magnetic field to the magnetostrictive member to cause the member to elongate;
a flexible structure coupled to the magnetostrictive member so as to change its lateral extent in response to elongation of the magnetostrictive member;
a first heat conductive structure attached to, and moving with, a lateral portion of the flexible structure; and
a second heat conductive structure located such that it is not in contact with the first heat conductive structure when the magnetostrictive member is not elongated and is in contact with the first heat conductive member when the magnetostrictive member is elongated.
